(19) United States
(12) Patent Application Publication
(10) Pub. No.: US 2018/0189018 A1
(43) Pub. Date: Jul. 5, 2018

(54) SOUNDING DEVICE HAVING NOTIFICATION FUNCTION, DATA ANALYSIS NOTIFICATION SYSTEM, AND DATA ANALYZING METHOD THEREOF

(71) Applicant: Unlimiter MFA Co., Ltd., Eden Island, SC (US)

(72) Inventors: Kuan-Li CHAO, Taipei (TW); Kuo-Ping YANG, Taipei (TW); Neo Bob Chih-Yung YOUNG, Taipei (TW)

(21) Appl. No.: 15/484,169

(22) Filed: Apr. 11, 2017

(30) Foreign Application Priority Data
Jan. 4, 2017 (TW) 106100183

(51) Int. Cl. G06F 3/16; G08B 3/10; G10L 13/00; A61B 5/01; A61B 5/021; A61B 5/00

(52) U.S. Cl. CPC G06F 3/16; G08B 3/10; G10L 13/00; A61B 5/7282; A61B 5/021; A61B 5/741; A61B 5/01

(57) ABSTRACT

A sounding device having a notification function, a data analysis notification system, and a data analyzing method the thereof are disclosed. The sounding device is used to establish a connection with an electronic device. The sounding device includes a sensing module, a data analysis module, a communication module, and a sounding module. The sensing module is used for sensing a sensing data. The data analysis module is used for analyzing the sensing data, wherein when the sensing data exceeds a threshold, a notice signal is generated. The communication module is used for transmitting the notice signal to the electronic device and receiving a voice signal returned from the electronic device, thus the sounding module is allowed to output the voice signal.

### BACKGROUND OF THE INVENTION

### 1. Field of the Invention

The present invention relates to a sounding device having notification function, a data analysis notification system, and a data analysis method thereof, particularly to a sounding device having notification function, a data analysis notification system, and a data analysis method thereof capable of detecting actively.

### 2. Description of the Related Art

With the advancement of technology, the usage of sounding devices such as earphones and speakers in modern society is increasing. The sounding device receives a sound signal directly from an electronic device to make a sound. In the prior art, the sounding device is passive to receive signals from the electronic device to make the sound. On the other hand, the prior art already has an electronic device that can detect a variety of signals, such as detecting the volume of the sound, the remaining amount of electricity, or the associated body information. These signals are detected actively by an electronic device, and then related voice signals are output through the sounding device. In this way, the sounding device can only be operated passively.

Therefore, it is necessary to invent a new sounding device having a notification function, a data analysis notification system and a data analysis method thereof to address the lack of prior art.

### SUMMARY OF THE INVENTION

It is a major objective of the present invention to provide a sounding device having notification function, which provides the effect of active sensing.

It is another objective of the present invention to provide a data analysis notification system used in the aforementioned sounding device.

It is a further objective of the present invention to provide a data analysis method used in the aforementioned system.

To achieve the objectives described above, the sounding device having notification function in the present invention is used for establishing a connection with an electronic device. The sounding device includes a sensing module, a data analysis module, a communication module, and a sounding module. The sensing module is used for sensing a sensing data. The data analysis module is electrically connected to the sensing module for analyzing the sensing data, wherein when the sensing data exceeds a threshold, the data analysis module generates a notice signal. The communication module is connected with the data analysis module and the electronic device for transmitting the notice signal to the electronic device and receiving a voice signal returned from the electronic device. The sounding module is electrically connected to the communication module for outputting the voice signal.

The data analysis notification system in the present invention includes a sounding device and an electronic device. The sounding device includes a sensing module, a data analysis module, a communication module, and a sounding module. The sensing module is used for detecting the sensing data. The data analysis module is electrically connected to the sensing module, for analyzing the sensing data, wherein when the sensing data exceeds a threshold, the data analysis module generates a notice signal. The communication module is electrically connected to the data analysis module and the electronic device for transmitting the notice signal to the electronic device and receiving a voice signal returned from the electronic device. The sounding module is electrically connected to the communication module. The electronic device is electrically connected to the communication module for receiving the notice signal. The electronic device includes a sound generation module for generating a voice signal according to the notice signal, to pass back to the sounding device, such that the sounding module outputs the voice signal.

A data analysis method according to the present invention includes the following steps: sensing a sensing data; analyzing the sensing data to conform if it exceeds a threshold; when the sensing data exceeds the threshold, a notice signal is generated; transmitting the notice signal to the electronic device and receiving a voice signal returned form the electronic device; and outputting the voice signal.

### BRIEF DESCRIPTION OF THE DRAWINGS

FIG. 1 is an architecture diagram of a data analysis notification system in the present invention; and

FIG. 2 is a flowchart showing steps of a data analysis method in the present invention.

### DETAILED DESCRIPTION OF THE PREFERRED EMBODIMENT

Hereafter, the technical content of the present invention will be better understood with reference to preferred embodiments.

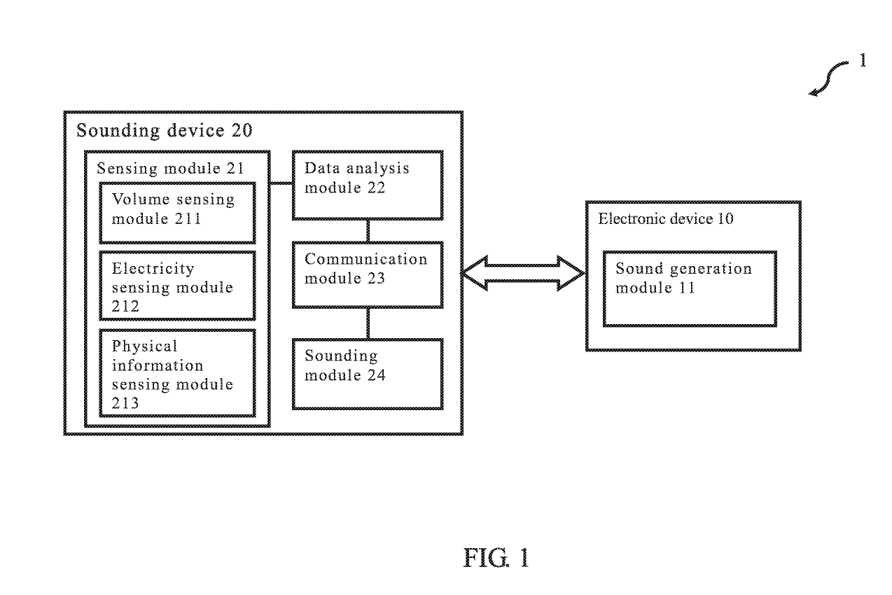

Hereafter please first refer to FIG. 1 which is an architecture diagram of a data analysis notification system in the present invention.

A data analysis notification system 1 of the present invention comprises an electronic device 10 and a sounding device 20. The electronic device 10 may be a desktop computer, a laptop, a smart phone, or a tablet, but the present invention is not limited thereto. The sounding device 20 may be an earphone or a speaker to be connected to the electronic device 10 in a wired or wireless manner means. The electronic device 10 may comprises a sound generation module 11. The sound generation module 11 can generate a high-definition voice signal according to a sound signal from the sounding device 20, which can be used to pass back to the sounding device 20.

The sounding device 20 includes a sensing module 21, a data analysis module 22, a communication module 23, and a sounding module 24. The data analysis module 22 may be a hardware circuit architecture or a hardware with software architecture, for actively sensing a sensing data. In one embodiment of the present invention, the sensing module 21 may comprise a volume sensing module 211, an electricity sensing module 212, and a physical information sensing module 213. The volume sensing module 211 is used for sensing an output volume information of the sounding module 24. In another embodiment of the present invention, the volume sensing module 211 can collect audio information outputted from the sounding module 24 to get the sound information. Besides volumes, the sound information also includes frequency, time, etc., but the present invention is not limited to those listed above. The electricity sensing module 212 can be used to sense the power of the sounding device 20 to obtain device power information. The physical information sensing module 213 is used for directly detecting a user who wears the sounding device 20 to obtain the user's physical information, such as a temperature sensing signal, a blood pressure sensing signal or a pulse sensor signal, but the present invention is not limited thereto.

The data analysis module 22 is electrically connected to the sensing module 22, for analyzing and determining whether the sensed information exceeds a threshold, for example, the output volume information detected by the volume sensing module 211 can be used for analyzing whether the volume of the sounding device 20 is excessively large and exceeds its previously set threshold. If the volume is too large to exceed the threshold, the data analysis module 22 will issue a notice signal containing the message code. At last, the communication module 23 is electrically connected to the electronic device 10 in a wired or wireless manner, for communicating with the electronic device 10 in two directions. The sounding module 24 may be an earphone or a speaker for outputting a sound signal which may be obtained by the electronic device or by an internal storage device (not shown) of the sounding device 20, but the present invention does not limit the source of the sound signal. After the data analysis module 22 analyzes and obtains a notice signal, the communication module 23 is used to transmit the notice signal to the electronic device 10. Since the message signal contains the message code, the sound generation module 11 of the electronic device 10 will generate a high-definition voice signal, and then the communication module 23 will send it back. At last, the sounding module 24 will output the voice signal, so that users clearly know the voice content. For example, when the original volume of the signal output by the sounding module 24 is too large, a voice signal generated by the sound generation module 11 of the electronic device 10 can be used to inform users clearly.

In addition, the sensing information obtained by the sensing module 22 can also be transmitted to the electronic device 10 through the communication module 23, for further confirming whether the sensing information exceeds a threshold. Then, similarly, the sound generation module 11 will generate a high-definition voice signal. Finally, the sounding module 24 outputs the voice signal to inform the user.

It should be noted that each of the modules in the electronic device 10 and sounding device 20 may be configured as hardware devices, software programs in combination with hardware device, or firmware in combination with hardware device, but the present invention is not limited in the manner described above. Additionally, the preferred embodiment of the present invention described here is only illustrative. To avoid redundancy, all the possible combinations of changes are not documented in detail. However, it shall be understood by those skilled in the art that each of the modules or elements described above may not be necessary. For the implementation of the present invention, the present invention may also contain other detailed, conventional modules or elements. Each module or component is likely to be omitted or modified depending on the needs. Other modules or elements may not necessarily exist between two of any modules.

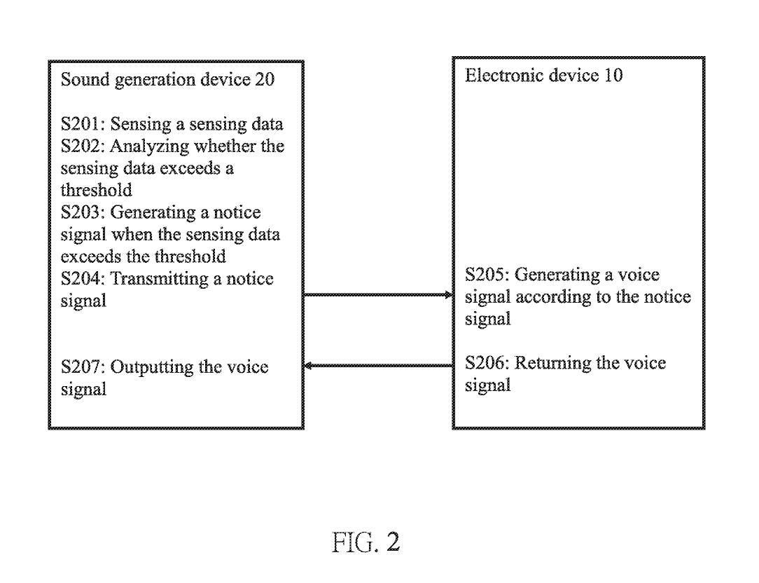

Then, please refer to FIG. 2 which is a flowchart showing steps of the data analysis method in the present invention. It should be noted here that although the above data analysis notification system 1 is used as an example to illustrate the data analysis method in the present invention, the data analysis method of the present invention is not limited to the data analysis notification system 1 that uses the same structure described above.

At first, Step S201 is executed: Sensing a sensing data.

First, the sensing module 21 is used for sensing a sensing data directly. For example, the volume sensing module 211 is used for sensing an output volume information of the sounding module 24, the electricity sensing module 212 is used for sensing the power of the sounding device 20 to obtain the device power information, or the physical information sensing module 213 is used for directly sensing a user who wears the sounding device 20 to obtain the user's physical information. However, the present invention is not limited to the embodiment described above.

Then, Step S202 is executed: analyzing whether the sensing data exceeds a threshold.

At this time, the data analysis module 22 is used for analyzing whether the sensed information exceeds a threshold.

Next Step S203 is executed: When the sensed data exceeds the threshold, a notice signal is generated.

If the sensed data exceeds the threshold, the data analysis module 22 will issue a notice signal containing message code.

Then, Step S204 is executed: Transmitting the notice signal.

After the data analysis module 22 analyzes and obtains the notice signal, the communication module 23 will be used for transmitting the notice signal to the electronic device 10.

With the electronic device 10, Step S205 is executed: Generating a voice signal according to the notice signal.

Since the notice signal contains message code, the sound generation module 11 of the electronic device 10 will generate a high-definition voice signal.

Next, Step S206 is executed: Returning the voice signal.

Then, the electronic device 10 returns the voice signal, such that the communication module 23 receives the returned signal.

Finally, Step S207 is executed: Outputting the voice signal.

At last, the sounding module 24 outputs the voice signal, such that user knows the voice content clearly.

It should be noted here that the data analysis method in the present invention is not limited to the order of the above steps. The order of the above steps may also be changed as long as the present invention can be achieved.

In this way, when the sounding device 20 is used, the sounding device 20 can take the initiative and be immediately informed of the occurrence of the situation for further control.

It should be noted that the preferred embodiment of the present invention described here is only illustrative. To avoid redundancy, all the possible combinations of changes are not documented in detail. However, it shall be understood by those skilled in the art that each of the modules or elements described above may not be necessary. For the implementation of the present invention, the present invention may also contain other detailed, conventional modules or elements. Each module or component is likely to be omitted or modified depending on the needs. Other modules or elements may not necessarily exist between two of any modules.

What is claimed is:

1. A sounding device having a notification function, used for establishing a connection with an electronic device, the sounding device comprising:
a sensing module, used for detecting a sensing data;
a data analysis module electrically connected to the sensing module for analyzing the sensing data, wherein when the sensing data exceeds a threshold, the data analysis module generates a notice signal;
a communication module connected with the data analysis module and the electronic device for transmitting the notice signal to the electronic device and receiving a voice signal returned from the electronic device; and
a sounding module electrically connected to the communication module for outputting the voice signal.

2. The sounding device having the notification function as claimed in claim 1, wherein the sounding device is an earphone.

3. The sounding device having the notification function as claimed in claim 2, wherein the sensing module has a volume sensing module for sensing an output volume information of the sounding module.

4. The sounding device having the notification function as claimed in claim 2, wherein the sensing module has an electricity sensing module for sensing a device power information of the sounding device.

5. The sounding device having the notification function as claimed in claim 2, wherein the sensing module has a physical information sensing module for sensing a user's physical information.

6. The sounding device having the notification function as claimed in claim 5, wherein the user's physical information comprises a temperature sensing signal, a blood pressure sensing signal, or a pulse sensor signal.

7. A data analysis notification system, comprising:
a sounding device, comprising:
a sensing module, used for sensing a sensing data;
a data analysis module electrically connected to the sensing module for analyzing the sensing data, wherein when the sensing data exceeds a threshold, a notice signal is generated by the data analysis module;
a communication module electrically connected to the data analysis module and the electronic device for transmitting the notice signal to the electronic device and receiving a voice signal returned from the electronic device; and
a sounding module electrically connected to the communication module; and
an electronic device electrically connected to the communication module for receiving the notice signal, the electronic device comprising:
a sound generation module, used for generating a voice signal according to the notice signal, to return to the sounding device, so that the sounding module outputs the voice signal.

8. The data analysis notification system as claimed in claim 7, wherein the sounding device is an earphone.

9. The data analysis notification system as claimed in claim 8, wherein the sensing module has a volume sensing module for sensing an output volume information of the output module.

10. The data analysis notification system as claimed in claim 8, wherein the sensing module has an electricity sensing module for sensing a device power information of the sounding device.

11. The data analysis notification system as claimed in claim 8, wherein the sensing module has a physical information sensing module for sensing a user's physical information.

12. The data analysis notification system as claimed in claim 11, wherein the user's physical information comprises a temperature sensing signal, a blood pressure sensing signal, or a pulse sensing signal.

13. The data analysis notification system as claimed in claim 7, wherein the electronic device is a smart phone.

14. A data analysis method, used in a sounding device used for establishing a connection with an electronic device; the method comprising the following steps:
sensing a sensing data;
analyzing the sensing data to determine whether the sensing data exceeds a threshold;
when the sensing data exceeds the threshold, generating a notice signal;
transmitting the notice signal to the electronic device and receiving a voice signal returned from the electronic device; and
outputting the voice signal.

15. The data analysis method as claimed in claim 14, further comprising the step of sensing an output volume information, a device power information, or a user's physical information.

16. The data analysis method as claimed in claim 15, wherein the user's physical information comprises a temperature sensing signal, a blood pressure sensing signal, or a pulse sensor signal.
